(19) United States
(12) Patent Application Publication
(10) Pub. No.: US 2014/0024102 A1
(43) Pub. Date: Jan. 23, 2014

(54) MICROBIAL FUEL CELL, FUEL AND MICROBES FOR SAID FUEL CELL, BIOREACTOR AND BIOSENSOR

(71) Applicants: Hideki Sakai, Tokyo (JP); Ryuhei Matsumoto, Tokyo (JP); Shuji Fujita, Tokyo (JP); Yoshio Goto, Tokyo (JP); Yuichi Tokita, Tokyo (JP)

(72) Inventors: Hideki Sakai, Tokyo (JP); Ryuhei Matsumoto, Tokyo (JP); Shuji Fujita, Tokyo (JP); Yoshio Goto, Tokyo (JP); Yuichi Tokita, Tokyo (JP)

(73) Assignee: Sony Corporation

(21) Appl. No.: 13/985,508

(22) PCT Filed: Feb. 24, 2012
(86) PCT No.: PCT/JP2012/055295

§ 371 Date: Aug. 14, 2013

(30) Foreign Application Priority Data
Feb. 24, 2011 (JP) 2011 037779
Feb. 16, 2012 (JP) 2012-031405

(51) Int. Cl. H01M 8/16; C12N 15/63

(52) U.S. Cl. CPC H01M 8/16; C12N 15/63
USPC 435/252.2; 435/252.3; 435/252.33; 435/252.34; 435/287.1; 435/289.1; 429/2; 568/852

(57) ABSTRACT

As a technique for increasing the output of a microbial fuel cell, a microbial fuel cell including a polyol such as glycerol as a fuel and using a microbe in which an enzyme that catalyzes a redox reaction has been introduced by genetic recombination on the side of a negative electrode is provided. By this microbial fuel cell, the velocity of the reaction can be increased to thereby give a high output by retaining a microbe in which an enzyme that catalyzes a redox reaction such as diaphorase has been introduced by genetic recombination on the side of the negative electrode.

### TECHNICAL FIELD

The present technique relates to a microbial fuel cell, a fuel and a microbe for a negative electrode for the cell, and a bioreactor and a biosensor. More specifically, the present technique relates to a microbial fuel cell and the like including a polyol as a fuel.

### BACKGROUND ART

A fuel cell has a structure in which a positive electrode (an air electrode) and a negative electrode (a fuel electrode) are facing each other through an electrolyte (a proton conductor). In the fuel cell, a fuel that has been fed to the negative electrode is oxidized to thereby be separated into electrons and protons (H⁺), and the electrons are transported to the negative electrode and the protons transfer to the positive electrode through the electrolyte. In the positive electrode, the protons react with oxygen that has been fed from outside and the electrons that have been sent from the negative electrode through an outer circuit to thereby form water (H₂O).

Focusing on that biological metabolism conducted in an organism is a high-efficient energy conversion mechanism, a suggestion for applying this to a fuel cell has been made. The biological metabolism as used herein includes aspiration conducted in cells and photonic synthesis, and the like. Biological metabolism gives an extremely high power generation efficiency, and the reaction thereof proceeds under a mild condition at about room temperature.

For example, aspiration takes nutrients such as saccharides, fats and proteins into a microbe or cells, and decomposes them stepwise by many enzymatic reaction steps. In the cases of saccharides, the chemical energy of the saccharide is converted to electrical energy in the process of generation of carbon dioxide (CO₂) through a glycolytic pathway or a tricarboxylic acid (TCA) circuit. Specifically, nicotinamide adenine dinucleotide ((NAD⁺) is reduced to thereby converted to reduced nicotinamide adenine dinucleotide (NADH), and these NADH are directly converted to electrical energy for a proton gradient and oxygen is reduced to thereby produce water.

As a technique for utilizing biological metabolism in a fuel cell, a microbial fuel cell by which an electrical current is obtained by taking electrons generated in a microbe out of the organism, and transmitting the electrons to an electrode was reported (for example, see Patent Document 1).

Furthermore, as a technique for utilizing biological metabolism in a fuel cell, a biofuel cell including a redox enzyme that is fixed as a catalyst on at least one electrode of a cathode or an anode has also been developed (for example, see Patent Documents 2 to 11). This biofuel cell separates protons and electrons by decomposing a fuel by using an enzyme as a catalyst, and those using alcohols such as methanol and ethanol or monosaccharides such as glucose as fuels have been developed. For example, in a biofuel cell including glucose as a fuel, as shown in FIG. 4A and FIG. 4B, an oxidation reaction of the glucose proceeds on a negative electrode and a reduction reaction of oxygen proceeds on a positive electrode. Currently, biofuel cells that can use not only glucose and oxygen but also various fuels have been gradually developed.

An electron mediator (an electron transmitting substance) is used on the positive electrodes and negative electrodes of these microbe fuel cells and biofuel cells for the purpose of smoothly transferring electrons between the microbe or enzyme used as a catalyst and the electrodes (for example, see Patent Documents 1 to 11).

### CITATION LIST

### Patent Documents

Patent Document 1: Japanese Patent Application Laid-Open (JP-A) No. 2000-133297
Patent Document 2: JP-A No. 2003-282124
Patent Document 3: JP-A No. 2004-71559
Patent Document 4: JP-A No. 2005-13210
Patent Document 5: JP-A No. 2005-310613
Patent Document 6: JP-A No. 2006-24555
Patent Document 7: JP-A No. 2006-49215
Patent Document 8 JP-A No. 2006-93090
Patent Document 9: JP-A No. 2006-127957
Patent Document 10: JP-A No. 2006-156354
Patent Document 11: JP-A No. 2007-12281
Patent Document 12: JP-A No. 2007-143493
Patent Document 13: JP-A No. 2008-289398
Patent Document 14: JP-A No. 2008-289419
Patent Document 15: JP-A No. 2008-48703
Patent Document 16: U.S. Patent application Laid-Open No. 2007/196899

### SUMMARY OF THE INVENTION

### Problems to be Solved by the Invention

There are many reactions other than reactions for converting chemical energy to electrical energy in the biological metabolism of a microbe. Therefore, microbial fuel cells have a problem that chemical energy is consumed by undesired reactions, and thus a sufficient energy conversion efficiency is not attained and the obtained output is much smaller than those obtained in biofuel cells. Furthermore, it is considered that the factors of the lower output of microbial fuel cells than that of biofuel cells are insufficient permeation of a substance (fuel or mediator) to the biomembrane of the microbe, a low velocity of an enzymatic reaction that is required for the cell output in the microbe, and the like.

Therefore, the present technique mainly aims at providing a technique for improving the output of a microbial fuel cell.

### Solutions to Problems

In order to resolve the above-mentioned problem, the present technique provides a microbial fuel cell including a polyol such as glycerol as a fuel.

In this microbial fuel cell, it is preferable to use a microbe in which an enzyme that catalyzes a redox reaction has been introduced by genetic recombination as the microbe. By such genetic recombination, the metabolism velocity of the fuel can be increased and thus a high output can be obtained. On the other hand, it is preferable to use a microbe from which an enzyme that is not involved in these reactions or an enzyme that inhibits the reaction has been deleted by genetic recombination. By such genetic recombination, reactions that do not contribute to the conversion of chemical energy to electrical energy can be suppressed and thus a high energy conversion efficiency can be obtained.

In this microbial fuel cell, the redox reaction can be a reaction for generating nicotinamide adenine dinucleotide (NAD⁺) or flavin adenine dinucleotide (FAD) by oxidizing a reduced coenzyme such as reduced nicotinamide adenine dinucleotide (NADH) and reduced flavin adenine dinucleotide (FADH₂), or a reaction for generating reduced nicotinamide adenine dinucleotide (NADH) or reduced flavin adenine dinucleotide (FADH₂) by reducing an oxidized coenzyme such as nicotinamide adenine dinucleotide (NAD⁺) and flavin adenine dinucleotide (FAD). In this case, the enzyme that catalyzes the redox reaction may be an enzyme such as diaphorase, which generates an oxidized coenzyme such as nicotinamide adenine dinucleotide (NAD⁺) and flavin adenine dinucleotide (FAD) by oxidizing a reduced coenzyme such as reduced nicotinamide adenine dinucleotide (NADH) and reduced flavin adenine dinucleotide (FADH₂).

Furthermore, the present technique provides a fuel for a microbial fuel cell including a polyol, and a microbe for a negative electrode for a microbial fuel cell in which an enzyme that catalyzes a redox reaction has been introduced by genetic recombination.

In addition, the present technique also provides a bioreactor and a biosensor, which include a microbe in which an enzyme that catalyzes a redox reaction has been introduced by genetic recombination.

The “microbial fuel cell” in the present technique encompasses a cell in which a microbe is retained on an electrode or the vicinity of the electrode, electrons generated in a fungus body are taken out of the fungus body by the metabolism of the fuel by the microbe, and the electrons are transferred to the electrode to thereby give an electrical current. In addition, the “microbial fuel cell” in the present technique also includes a cell in which an enzyme that is generated by a microbe and secreted toward outside of the fungus body is fed onto an electrode or to the vicinity of the electrode, and electrons are taken out by an oxidation reaction of a fuel using this enzyme as a catalyst to thereby give an electrical current.

### Effects of the Invention

According to the present technique, a technique for increasing the output of a microbial fuel cell is provided.

### BRIEF DESCRIPTION OF DRAWINGS

FIG. 1 is a cross-sectional schematic view for the explanation on the constitution of the microbial fuel cell according to the present technique.

FIG. 2 is a drawing for the explanation on a system for measuring the number of electrons in an oxidation reaction of glycerol by Escherichia coli (Example 1).

FIG. 3 is a graph as a substitute for a drawing, which shows the result of the measurement of the number of electrons in an oxidation reaction of glycerol by Escherichia coli (Example 1).

FIG. 4A and FIG. 4B are drawings for the explanation on a redox reaction at an electrode of a biofuel cell including glucose as a fuel.

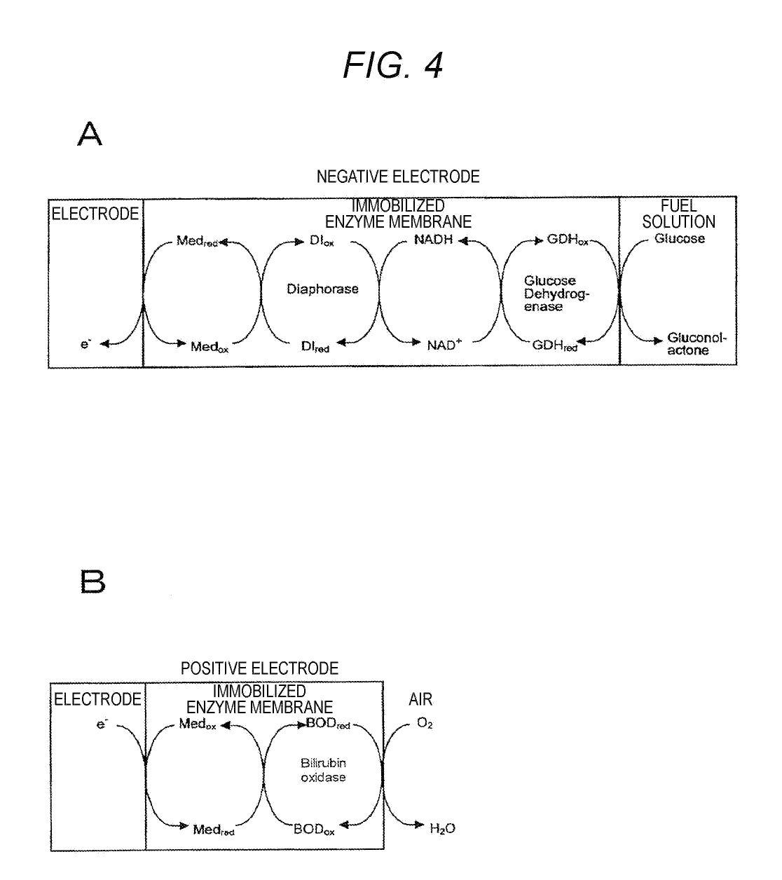

### MODE FOR CARRYING OUT THE INVENTION

The following is an explanation on preferable embodiments for carrying out the present technique, with reference to the drawings. The embodiments explained below show examples of typical embodiments of the present technique, and the scope of the present technique is not interpreted to be narrow by these embodiments. Explanation will be made in the following order.

1. Microbial fuel cell
(1) Structure of cell

### (2) Microbe

### (3) Fuel

(4) Electrode material
(5) Negative electrode enzyme
(6) Positive electrode enzyme
(7) Proton conductor

### 2. Bioreactor

1. Microbial Fuel Cell

(1) Structure of Cell

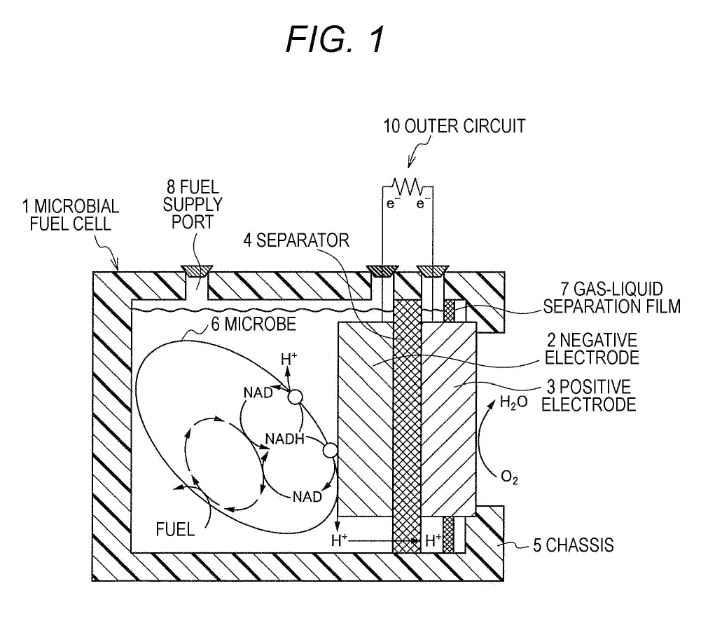

FIG. 1 schematically shows the constitution of the microbial fuel cell according to the present technique. The microbial fuel cell shown by Symbol 1 includes a counter electrode composed of a negative electrode 2 and a positive electrode 3, a separator 4 that is configured to separate the counter electrode, and a chassis 5 that is configured to house these. The negative electrode 2 and positive electrode 3 are electrically connected by an outer circuit 10. A proton conductor is housed in the chassis 5. The separator 4 is formed of, for example, a material that can allow the permeation of protons such as a cation exchange membrane, a cellulose-based woven fabric and cellophane.

The negative electrode 2 takes out electrons by an oxidation reaction of a fuel. The fuel and a microbe 6 are retained on the side of the negative electrode 2 in the state that they are required for power generation. On the negative electrode 2, the fuel is oxidized and decomposed by utilizing the biological metabolism of the microbe 6 in the catalyzing process, and a reaction of taking out the electrons proceeds.

A part of the positive electrode 3 is exposed to the outside of the chassis 5 from a gas-liquid separation film 7. On the positive electrode 3, a reduction reaction of oxygen that is fed from outside proceeds.

The protons that have been separated together with the electrons from the fuel at the side of the negative electrode 2 permeate the separator 4 and transfer to the side of the positive electrode 3. The protons that have transferred to the side of the positive electrode 3 each receives an electron from the positive electrode 3 and binds to oxygen to thereby form water.

(2) Microbe

The microbe 6 that is retained on the side of the negative electrode 2 is a microbe in which an enzyme that catalyzes a redox reaction has been introduced by genetic recombination, and is considered to be a microbe that expresses the enzyme more than a wild-type microbe does. Furthermore, the microbe 6 is preferably a microbe from which an enzyme that is not involved in a redox reaction or an enzyme that inhibits the reaction has been deleted by genetic recombination.

The redox reaction as used herein refers to a series of reactions including an oxidation reaction in the process of decomposing the fuel by the microbe, and a reaction in which a reduced form of a coenzyme (for example, NADH, NADPH and the like) is generated from a coenzyme (for example, NAD⁺, NADP⁺ and the like) in accordance with this oxidation reaction, and the reduced form of the coenzyme is oxidized by a coenzyme oxidase (for example, diaphorase) to thereby generate electrons.

The coenzyme also includes flavin adenine dinucleotide (FAD⁺), pyrrollo-quinoline quinone (PQQ²⁺) and the like.

Of the enzymes that catalyze the above-mentioned reaction, examples of enzymes that catalyze an oxidation reaction in the process of decomposition of the fuel may include the following enzymes. Glucose dehydrogenase, gluconate 5-dehydrogenase, gluconate 2-dehydrogenase, alcohol dehydrogenase, aldehyde reductase, aldehyde dehydrogenase, lactate dehydrogenase, hydroxyparuvate reductase, glycerate dehydrogenase, formate dehydrogenase, fructose dehydrogenase, galactose dehydrogenase, malic acid dehydrogenase, glyceraldehyde 3-phosphate dehydrogenase, lactic acid dehydrogenase, sucrose dehydrogenase, fructose dehydrogenase, sorbose dehydrogenase, pyruvate dehydrogenase, isocirate dehydrogenase, 2-oxoglutarate dehydrogenase, succinate dehydrogenase, maleate dehydrogenase, acyl-CoA dehydrogenase, L-3-hydroxyacyl-CoA dehydrogenase, 3-hydroxypropionate dehydrogenase, 3-hydroxybutyrate dehydrogenase and the like.

Furthermore, of the enzymes that catalyze the above-mentioned reaction, examples of coenzyme oxidases that catalyze a reaction to generate electrons by oxidizing a reduced form of a coenzyme may include diaphorase and the like, which catalyze a reaction for generating nicotinamide adenine dinucleotide (NAD⁺) or flavin adenine dinucleotide (FAD) by oxidizing reduced nicotinamide adenine dinucleotide (NADH) or reduced flavin adenine dinucleotide (FADH₂). The enzymes listed above may be mutant enzymes whose catalytic activities have been improved by genetic modification (see Patent Documents 12 to 16).

The enzyme that is not involved in the above-mentioned reaction or the enzyme that inhibits the reaction may include a series of enzymes that are involved in metabolism reactions of pathways that do not bind to the respiratory chains in the metabolism pathways of the microbe. Examples include enzyme groups that are involved in only the synthesis of substances such as pyrimidine, amino acids, ketone bodies, cholesterol, glycogen, phospholipids, triglycerides and purine, and the like. Furthermore, enzyme groups that are involved in only the decomposition of nucleic acids may also be exemplified.

Examples of the microbe may include facultative anaerobic bacteria such as those belonging to various genera such as Escherichia, Shigella, Salmonella, Citrobacter, Klebsiella, Enterobacter, Erwinia, Serratia, Hafnia, Edwardsiella, Proteus, Providencia, Morganella, Yersinia, Obesumbacterium, Xenorhabdus, Kluyvera, Rahnella, Cedecea, Tatumella, Vibrio, Photobacterium, Aeromonas, Plesiomonas, Pasteurella, Haemophilus, Actinobacillus, Zymomonas, Chromobacterium, Cardiobacterium, Calymmatobacterium, Gardnerella, Eikenella and Streptobacillus. Furthermore, strictly anaerobic bacteria such as those belonging to various genera such as Bacteroides, Fusobacterium, Leptotrichia, Butyrivibrio, Succinimonas, Succinivibrio, Anaerobiospirillum, Wolinella, Selenomonas, Anaerovibrio, Pectinatus, Acetivibrio and Lachnospira may be exemplified.

In addition, strictly aerophilic bacteria such as those belonging to various genera such as Pseudomonas, Xanthomonas, Frateuria, Zoogloea, Azotobacter, Azomonas, Rhizobium, Bradyrhizobium, Agrobacterium, Phyllobacterium, Methylococcus, Methylomonas, Halobacterium, Halococcus, Acetobacter, Gluconobacter, Legionella, Neisseria, Moraxella, Acinetobacter, Kingella, Beijerinckia, Derxia, Xanthobacter, Thermus, Thermomicrobium, Halomonas, Alteromonas, Flavobacterium, Alcaligenes, Serpens, Janthinobacterium, Brucella, Bordetella, Francisella, Paracoccus and Lampropedia may be exemplified.

Furthermore, microaerophilic bacteria such as those belonging to various genera such as Aquaspirillum, Spirillum, Asospirillum, Oceanospirillum, Campylobacter, Bdellovibrio and Vampirovibrio may be exemplified.

Among these, anaerobic bacteria are preferable. This is because the negative electrode 2 is maintained under an anaerobic condition so that the electrons that have been taken out would not be consumed by the reaction with oxygen.

An enzyme (a recombinant enzyme) can be introduced into the microbe by genetic recombination by using a conventionally-known means. A recombinant enzyme gene can be inserted into a vector (a plasmid) by using a commercially available ligation kit or the like. As a method for introducing the obtained vector into a host, for example, a method including treating competent cells with calcium chloride or the like may be used.

Furthermore, deletion (or inactivation) of the microbe gene by genetic recombination can be conducted by a non-genetic engineering means by mutation by using a mutagen, a genetic engineering means by arbitrarily manipulating gene sequences by using a restriction enzyme or ligase or the like, or the like. As the method for deleting by a genetic engineering means, a method including preparing a DNA including a mutant gene by cloning an enzyme gene in advance and causing mutation on a specific site of the gene by a non-genetic engineering means or a genetic engineering means, disposing a deleted site with a specific length on a specific site of the gene by a genetic engineering means, or introducing an exogenous gene such as a drug resistant marker in a specific site of the gene, or the like, and returning this DNA to the microbe, is used.

The microbe 6 can be retained on the side of the negative electrode 2 in the state required for power generation, by being incorporated in the solution on the side of the negative electrode 2. Alternatively, the microbe 6 can be retained on the side of the negative electrode 2 by being attached or fixed on a support or on the negative electrode 2. As the support, many microbial supports that are utilized in pharmaceutical industries and food industries, or bioreactors such as drainage treatment systems can be used. Specifically, for example, particular supports such as porous glass, ceramics, metal oxides, active carbon, kaolinite, bentonite, zeolite, silica gel, alumina and anthracite, gel-like supports such as starch, agar, chitin, chitosan, polyvinyl alcohol, alginic acid, polyacrylamide, carrageenan, agarose and gelatin, polymer resins such as cellulose, glutalaldehyde, polyacrylic acid and urethane polymer, ion exchange resins and the like are used. Furthermore, natural or synthetic polymer compounds such as cotton, hemp, pulp materials, or polymeric acetates obtained by modifying natural substances, polyesters and polyurethanes can also be utilized.

In the microbial fuel cell 1, the velocity of the above-mentioned reaction can be increased by using the microbe 6 in which an enzyme that catalyzes a redox reaction has been introduced by genetic recombination on the side of the negative electrode 2. Therefore, a higher output than before can be obtained in the microbial fuel cell 1. Furthermore, in the microbial fuel cell 1, reactions that do not contribute to the conversion of chemical energy to electrical energy can be suppressed to thereby prevent consumption of electrical energy by using the microbe 6 from which an enzyme that is not involved in a redox reaction or an enzyme that inhibits the reaction has been deleted by genetic recombination on the side of the negative electrode 2. Therefore, a higher energy conversion efficiency than before can be obtained in the microbial fuel cell 1.

(3) Fuel

The fuel retained on the side of the negative electrode 2 is not specifically limited as long as it is a substance that can be a nutrient for the microbe 6. Examples of the substance that can be used as the fuel may include saccharides, alcohols, aldehydes, lipids or proteins, and the like. Specific examples may include saccharides such as glucose, fructose, sorbose, starch, amylose, amylopectin, glycogen, cellulose, maltose, sucrose and lactose, alcohols such as ethanol and glycerin, organic acids such as acetic acid and pyruvic acid, and the like. Other examples may include fats, proteins, and organic acids that are intermediate products of the sugar metabolism of these, and the like.

As mentioned below in Examples, the present inventors first revealed that a microbe that had been considered to be impossible to metabolite glycerol under an anaerobic condition in the past can drive polyol metabolism by conjugating an electrochemical oxidation system through an electron transfer mediator. This polyol metabolism was such that electrons can be taken out by oxidizing glycerol with a high efficiency. Therefore, a polyol can be specifically adopted as the fuel. Examples of the polyol may include trihydric polyhydric alcohols such as glycerol, dihydric polyhydric alcohols such as ethylene glycol, and the like. Among these, specifically for glycerol, effective utilizations of glycerol that is generated as a by-product of biodiesel have been sought in recent years. Utilization of glycerol as a fuel for a microbial fuel cell can be one of the effective utilizations.

(4) Electrode Material

As the material for the negative electrode 2 and positive electrode 3, carbon-based materials such as porous carbon, carbon pellets, carbon paper, carbon felt, carbon fibers or laminates of carbon microparticles can be preferably adopted. Furthermore, the following metal materials can also be adopted as the material for the negative electrode 2 and positive electrode 3. Metals such as Pt, Ag, Au, Ru, Rh, Os, Nb, Mo, In, Ir, Zn, Mn, Fe, Co, Ti, V, Cr, Pd, Re, Ta, W, Zr, Ge, Hf. Alloys such as alumel, brass, duralumin, bronze, nickelin, platinum-rhodium, Hyperco, permalloy, permendur, German silver and phosphor bronze. Borides such as HfB₂, NbB and CrB₂. Nitrides such as TiN and ZrN. Silicides such as VSi₂, NbSi₂, MoSi₂ and TaSi₂. Composite materials of these.

(5) Negative Electrode Enzyme

An electron transfer mediator for smooth transfer of the electrons that have been taken out by the microbe 6 to the electrode may be fixed on the negative electrode 2. Although various materials can be used as the electron transfer mediator, it is preferable to use a compound having a quinone backbone or a compound having a ferrocene backbone. As the compound having a quinone backbone, benzoquinones, or compounds having a naphthoquinone backbone or an anthraquinone backbone are specifically preferable.

As the benzoquinones, 2,3-dimethoxy-5-methyl-1,3-benzoquinone (Q₀)) and the like can be used.

As the compounds having a naphthoquinone backbone, for example, 2-amino-1,4-naphthoquinone (ANQ), 2-amino-3-methyl-1,4-naphthoquinone (AMNQ), 2-amino-3-carboxy-1,4-naphthoquinone (ACNQ), 2,3-diamino-1,4-naphthoquinone, 4-amino-1,2-naphthoquinone, 2-hydroxy-1,4-naphthoquinone, 2-methyl-3-hydroxy-1,4-naphthoquinone, vitamin K₁ (2-methyl-3-phytyl, 4-naphthoquinone), vitamin K₂(2-farnesyl-3-methyl-1,4-naphtoquinone), vitamin K₃ (2-methy 1,4-naphthoquinone) and the like can be used.

Furthermore, as the compounds having a quinone backbone, for example, compounds having an anthraquinone backbone such as anthraquinone-1-sulfonate and anthraquinone-2-sulfonate, and derivatives thereof can be used.

Furthermore, as the compound having a ferrocene backbone, for example, vinylferrocene, dimethylaminomethylferrocene, 1,1′-bis(diphenylphosphino)ferrocene, dimethylferrocene, ferrocenemonocarboxylic acid and the like can be used.

Furthermore, as the other compounds, for example, metal complexes of iron (Fe), compounds having a nicotinamide structure, compounds having a riboflavin structure, compounds having a nucleotide-phosphate structure and the like can be used. More specifically, for example, methylene blue, pycocyanine, indigo-tetrasulfonate, luciferin, gallocyanine, pyocyanine, methyl apri blue, resorufin, indigo-trisulfonate, 6,8,9-trimethyl-isoalloxazine, chloraphine, indigo disulfonate, nile blue, indigocarmine, 9-phenyl-isoalloxazine, thioglycolic acid, 2-amino-N-methyl phenazinemethosulfate, azure A, indigo-monosulfonate, anthraquinone-1,5-disulfonate, alloxazine, brilliant alizarin blue, crystal violet, patent blue, 9-methyl-isoalloxazine, cibachron blue, phenol red, anthraquinone-2,6-disulfonate, neutral blue, bromphenol blue, anthraquinone-2,7-disulfonate, quinoline yellow, riboflavin, Flavin mononucleotide (FMN), flavin adenine dinucleotide (FAD), phenosafranin, lipoamide, safranine T, lipoic acid, indulin scarlet, 4-aminoacridine, acridine, nicotinamideadenine dinucleotide (NAD), nicotinamide adenine dinucleotide phosphate (NADP), neutral red, cysteine, benzyl viologen(2+/1+), 3-aminoacridine, 1-aminoacridine, methyl viologen(2+/1+), 2-aminoacridine, 2,8-diaminoacridine, 5-aminoacridine and the like can be used. In the chemical formulas, dien represents diethylenetriamine, and edta represents ethylenediaminetetraacetate tetraanione, respectively.

(6) Positive Electrode Enzyme

An enzyme that catalyzes a reduction reaction of oxygen that is fed from the outside exists on the positive electrode 3. Examples of such enzyme may include laccase, bilirubin oxydase, ascorbate oxydase, CueO, CotA and the like.

Furthermore, an electron transfer mediator for smooth transfer of the electrons that have been sent from the negative electrode 2 may be fixed on the positive electrode 3. The electron transfer mediator that can be fixed on the positive electrode 3 preferably has a higher redox potential than that of the electron transfer mediator that is used for the negative electrode 2.

Specifically, ABTS (2,2′-azinobis(3-ethylbenzoline-6-sulfonate)), K₃[Fe(CN)₆], Cu^{III/II} (H₂A₃)^{0/1-}, [Fe(dpy)]^{3+/2+}, Cu^{III/II} (H₂G₃a)^{0/1-}, I₃−/I−, ferrocene carboxylic acid, [Fe(CN)₆]^{3-/4-}, ferrocene ethanol, Fe^{3+/2+}, malonate, Fe^{3+/2+}, salycylate, [Fe (edta)]^{1-/2-}, [Fe(ox)₃]^{3-/4-}, promazine (n=1) [ammonium form], chloramine-T, TMPDA (N,N,N′,N′-tetramethylphenylenediamine), porphyrexide, syringaldazine, o-tolidine, bacteriochlorophyll a, dopamine, 2,5-dihydroxy-1,4-benzoquinone, p-amino-dimethylaniline, o-quinone/1,2-hydroxybenzene (catechol), p-aminophenoltetrahydroxy-p-benzoquinone, 2,5-dichloro-p-benzoquinone, 1,4-benzoquinone, diaminodurene, 2,5-dihydroxyphenylacetic acid, 2,6,2′-trichloroindophenol, indophenol, o-toluidine blue, DCPIP (2,6-dichlorophenolindophenol), 2,6-dibromo-indophenol, phenol blue, 3-amino-thiazine, 1,2-napthoquinone-4-sulfonate, 2,6-dimethyl-p-benzoquinone, 2,6-dibromo-2′-methoxy-indophenol, 2,3-dimethoxy-5-methyl-1,4-benzoquinone, 2,5-dimethyl-p-benzoquinone, 1,4-dihydroxy-naphthoic acid, 2,6-dimethyl-indophenol, 5-isopropyl-2-methyl-p-benzoquinone, 1,2-naphthoquinone, 1-naphthol-2-sulfonate indophenol, toluoylene blue, TTQ (tryptophan tryptophylquinone) model (3-methyl-4-(3′-methylindol-2′-yl)indol-6,7-dione), ubiquinone (coenzyme Q), PMS (N-methylphenazinium methosulfate), TPQ (topa quinone or 6-hydroxydopa quinone), PQQ (pyrroloquinolinequinone), thionine, thionine-tetrasulfonate, ascorbic acid, PES (phenazine ethosulphate), cresyl blue, 1,4-naphthoquinone, toluidine blue, thiazine blue, gallocyanine, thioindigo disulfonate, methylene blue, vitamin K3 (2-methyl-1,4-naphthoquinone) and the like can be used. In the chemical formulas, dpy represents 2,2′-dipyridine, phen represents 1,10-phenanthroline, Tris represents tris(hydroxymethyl)aminomethane, trpy represents 2,2′:6′,2″-terpyridine, Im represents imidazole, py represents pyridine, thmpy represents 4-(tris(hydroxymethyl)methyl)pyridine, bhm represents bis(bis(hydroxymethyl)methyl, G3a represents triglycineamide, A3 represents trialanine, ox represents oxalate dianione, edta represents ethylenediaminetetraacetate tetraanione, gly represents glycinate anion, pdta represents propylenediaminetetraacetate tetraanione, trdta represents trimethylenediaminetetraacetate tetraanione, cydta represents 1,2-cyclohexanediaminetetraacetate tetraanione, respectively.

(7) Proton Conductor

As the proton conductor, an electrolyte solution (electrolyte liquid) that has no electron conductivity and can transport protons is used. As the electrolyte liquid, a neutral buffer at around pH 7 is specifically preferably used. As buffer substances, dihydrogen phosphate ion (H₂PO₄⁻) formed by sodium dihydrogen phosphate (NaH₂PO₄), potassium dihydrogen phosphate (KH₂PO₄) and the like, 2-amino-2-hydroxymethyl-1,3-propanediol (abbreviation: tris), 2-(N-morpholino)ethanesulfonic acid (MES), cacodylic acid, carbonic acid (H₂CO₃), hydrogen citrate ion, N-(2-acetamide)iminodiacetate (ADA), piperazine-N,N′-bis(2-ethanesulfonic acid) (PIPES), N-(2-acetamide)-2-aminoethanesulfonic acid (ACES), 3-(N-morpholino)propanesulfonic acid (MOPS), N-2-hydroxyethylpiperazine-N′-2-ethanesulfonic acid (HEPES), N-2-hydroxyethylpiperazine-N′-3-propanesulfonic acid (HEPES), N-[tris(hydroxymethyl)methyl]glycine (abbreviation: tricine), glycylglycine, N,N-bis(2-hydroxyethyl)glycine (abbreviation: bicine), imidazole, triazole, pyridine derivatives, bipyridine derivatives, compounds having an imidazole ring such as imidazole derivatives (histidine, 1-methylimidazole, 2-methylimidazole, 4-methylimidazole, 2-ethylimidazole, ethyl imidazole-2-carboxylate, imidazole-2-carboxyaldehyde, imidazole-4-carboxylic acid, imidazole-4,5-dicarboxylic acid, imidazol-1-yl-acetic acid, 2-acetylbenzimidazole, 1-acetylimidazole, N-acetylimidazole, 2-aminobenzimidazole, N-(3-aminopropyl)imidazole, 5-amino-2-(trifluoromethyl)benzimidazole, 4-azabenzimidazole, 4-aza-2-mercaptobenzimidazole, benzimidazole, 1-benzylimidazole, 1-butylimidazole), and the like can be used. Furthermore, Nafion (registered trademark), which is a solid electrolyte, can also be used.

2. Bioreactor and Biosensor

The microbe 6 used in the above-mentioned microbial enzyme cell 1 can also be applied to a bioreactor in which a biochemical reaction is conducted by using a biological catalyst, a biosensor that detects a substance by the substrate-specific change of the substance by the biochemical reaction, and the like. Such bioreactor or biosensor includes a reaction element including a microbial support and a microbe that is attached or fixed thereon as a basic constitution.

By using the microbe 6 in which an enzyme that catalyzes a reaction of taking out electrons from a substance has been introduced by genetic recombination in the reaction element, the velocity of a biochemical reaction can be increased. Therefore, in the above-mentioned bioreactor or biosensor, a desired substance can be reacted at a higher reaction velocity and a desired substance can be detected at a higher sensitivity than before. Furthermore, by using the microbe 6 from which an enzyme that is not involved in a reaction of taking out electrons from a substance or an enzyme that inhibits the reaction has been deleted by genetic recombination in the reaction element, reactions that do not contribute to a desired biochemical reaction can be suppressed, and thus higher reaction efficiency and detection sensitivity than before can be obtained.

The microbial fuel cell according to the present technique can also have the following constitution.

(1) A microbial fuel cell including a polyol as a fuel.
(2) The microbial fuel cell according to the above-mentioned (1), wherein the polyol is glycerol.
(3) The microbial fuel cell according to the above-mentioned (1) or (2), including a microbe in which an enzyme that catalyzes a redox reaction has been introduced by genetic recombination.
(4) The microbial fuel cell according to any of the above-mentioned (1) to (3), wherein the microbe is a microbe from which an enzyme that is not involved in a redox reaction or an enzyme that inhibits the reaction has been deleted by genetic recombination.
(5) The microbial fuel cell according to the above-mentioned (3) or (4), wherein the redox reaction is a reaction for generating a redox form of a coenzyme, and is any of a reaction for generating nicotinamide adenine dinucleotide (NAD⁺) by oxidizing reduced nicotinamide adenine dinucleotide (NADH), a reaction for generating reduced nicotinamide adenine dinucleotide (NADH) by reducing nicotinamide adenine dinucleotide (NAD⁺), a reaction for generating flavin adenine dinucleotide (FAD) by oxidizing reduced flavin adenine dinucleotide (FADH₂), or a reaction for generating reduced flavin adenine dinucleotide (FADH₂) by reducing flavin adenine dinucleotide (FAD).
(6) The microbial fuel cell according to above-mentioned (3) or (5), wherein the enzyme that catalyzes the redox reaction is diaphorase that catalyzes a reaction for generating nicotinamide adenine dinucleotide (NAD⁺) by oxidizing reduced nicotinamide adenine dinucleotide (NADH).

### EXAMPLES

### Example 1

### 1. Evaluation of Number of Electrons in Oxidization Reaction of Glycerol by Escherichia coli

The numbers of electrons in oxidization reactions of glycerol by wild-type Escherichia coli and mutant Escherichia coli under an anaerobic condition were measured by coulometry.

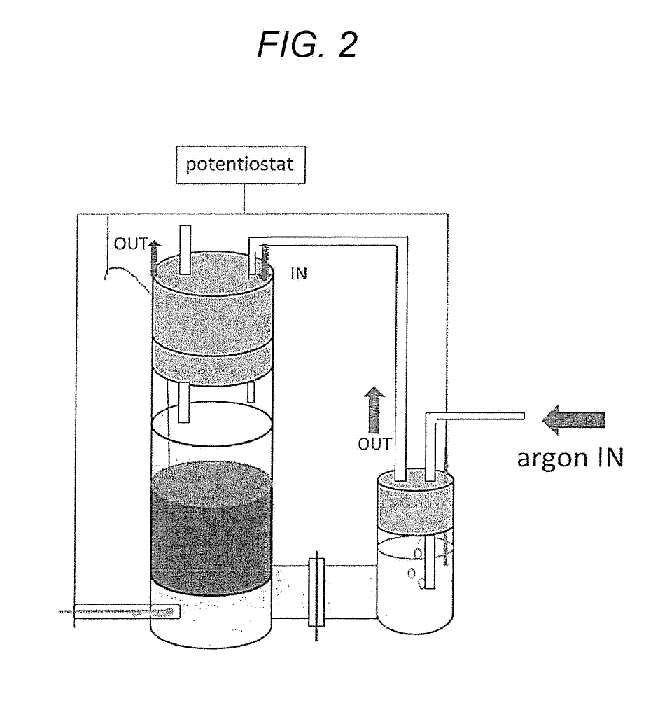

The conditions for the measurement were as follows, and the measurement was conducted as shown in the measurement drawing shown in FIG. 2.

Anaerobic Condition

Measurement temperature: 37° C.

Measurement cell: large cell for entire electrolysis (200 ml volume)

Working electrode: carbon felt (6 cm×14 cm)

Reference electrode: silver/silver chloride

Counter electrode: platinum wire

Applied potential: 0.4 V

Buffer: pH 8.0, M9 minimum culture medium 150 ml

Microbe: wild-type Escherichia coli (E. coli BL21 (DE3), mutant Escherichia coli (E. coli BL21 (DE3) pET12a-di Novagen), 1×10¹⁰ cell/ml

Glycerol concentration: 10 mM

Mediator: 2,3-Dimethoxy-5-methyl-1,3-benzoquinone (Q₀) 100 μM

Mutant Escherichia coli in which diaphorase had been genetically introduced was prepared according to the following procedure. A vector that expresses wild-type diaphorase derived from Bacillus stearothermophilus having the amino acid sequence shown in SEQ ID NO: 1 was constructed. An amplified fragment of a wild-type diaphorase gene was treated with BamH I and Nde I, and purified by using PCR Cleanup Kit (Qiagen). Furthermore, vector pET12a (Novagen) was treated with BamH I and Nde I, and purified in a similar manner. These two kinds of fragments were subjected to ligation by T4 ligase. The prepared vector was introduced in E. coli BL21 (DE3) by a heat shock process to thereby conduct transformation. The transformant was pre-cultured in SOC for 1 hr at 37° C. and thereafter spread on an LB-amp agar culture medium to give a colony, and a part of the colony was liquid-cultured, and the expression of diaphorase was confirmed by SDS-PAGE.

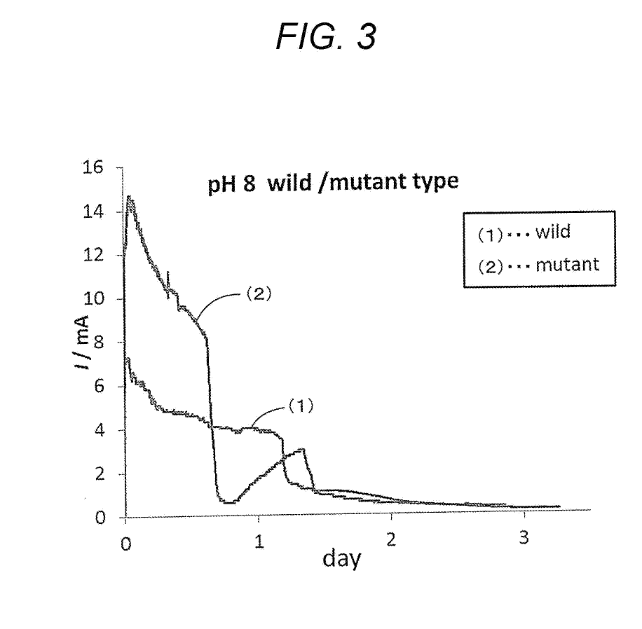

The result is shown in FIG. 3. For the wild-type Escherichia coli and mutant Escherichia coli, the quantity of electricity obtained when only the bacterium and mediator were used without adding glycerol was subtracted from the quantity of electricity obtained when glycerol was added, and the number of the oxidized electrons in the glycerol was calculated from the obtained difference by the following formula.

Q(difference in quantities of electricity)=n(number of electrons)·F(Faraday constant)·N(amount of substance)

As a result of the calculation, it was found that about four electrons were oxidized by the wild-type Escherichia coli and about five electrons were oxidized by the mutant Escherichia coli in the glycerol. This corresponds to an electrolysis efficiency of about 30% in the wild-type Escherichia coli and an electrolysis efficiency of about 40% in the mutant Escherichia coli with respect to a theoretical value in the case when glycerol is completely oxidized to CO₂. The reason why the electrolysis efficiency was increased more in the mutant Escherichia coli than in the wild-type Escherichia coli is considered that the redox reaction of NADH and Q₀, which had become a rate-controlling step, was eliminated by the gene transfection of diaphorase. Furthermore, the reasons why the efficiency was lower than 100% were presumed to be the growth of the bacteria, the unquantified metabolite, and that the electrons had transferred to the trace amount of oxygen remaining in the cell and the like.

### Example 2

### 2. Evaluation of Amount of Consumed Glycerol

NAD⁺ and glycerol dehydrogenase were added to the sample that had been collected over time from the measurement cell during the potentiostatic electrolysis, the amount of the generated NADH was obtained from the change in absorbance at 340 nm, and the amount of the consumed glycerol was evaluated.

The composition of the solution was as shown below.

pH10 NaHCO₃/NaOH buffer: 1 ml

1 M ammonium sulfate solution: 30 μl

10 mM NAD⁺ solution: 100 μl

Sample: 126 μl

30 μl of a glycerol dehydrogenase solution (Cellulomonas sp., SIGMA ALDRICH) dissolved at about 300 U/ml to the above-mentioned buffer

The change in absorbance at 340 nm was obtained, and the glycerol concentration in the sample was calculated based on a calibration curve; as a result thereof, the amount of the glycerol in the sample decreased in accordance with the decay of the electrical current and drained in about 20 hours in the case when either of the wild-type Escherichia coli and mutant Escherichia coli was used.

### INDUSTRIAL APPLICABILITY

The microbial fuel cell according to the present technique is useful as a microbial fuel cell whose output has been increased than before.

### REFERENCE SIGNS LIST

1 Microbial fuel cell
2 Negative electrode
3 Positive electrode
4 Separator
5 Chassis
6 Microbe
7 Gas-liquid separation film
8 Fuel supply port

1. A microbial fuel cell comprising a polyol as a fuel.

2. The microbial fuel cell according to claim 1, wherein the polyol is glycerol.

3. The microbial fuel cell according to claim 2, comprising a microbe in which an enzyme that catalyzes a redox reaction has been introduced by genetic recombination.

4. The microbial fuel cell according to claim 3, wherein the microbe is a microbe from which an enzyme that is not involved in a redox reaction or an enzyme that inhibits the reaction has been deleted by genetic recombination.

5. The microbial fuel cell according to claim 4, wherein the redox reaction is a reaction for generating a redox form of a coenzyme, and is any of a reaction for generating nicotinamide adenine dinucleotide (NAD⁺) by oxidizing reduced nicotinamide adenine dinucleotide (NADH), a reaction for generating reduced nicotinamide adenine dinucleotide (NADH) by reducing nicotinamide adenine dinucleotide (NAD⁺), a reaction for generating flavin adenine dinucleotide (FAD) by oxidizing reduced flavin adenine dinucleotide (FADH₂), or a reaction for generating reduced flavin adenine dinucleotide (FADH₂) by reducing flavin adenine dinucleotide (FAD).

6. The microbial fuel cell according to claim 5, wherein the enzyme that catalyzes the redox reaction is diaphorase that catalyzes a reaction for generating nicotinamide adenine dinucleotide (NAD⁺) by oxidizing reduced nicotinamide adenine dinucleotide (NADH).

7. A fuel for a microbial fuel cell comprising a polyol.

8. A microbe for an electrode for a microbial fuel cell in which an enzyme that catalyzes a redox reaction has been introduced by genetic recombination.

9. A bioreactor comprising a microbe in which an enzyme that catalyzes a redox reaction has been introduced by genetic recombination.

10. A biosensor comprising a microbe in which an enzyme that catalyzes a redox reaction has been introduced by genetic recombination.


SEQUENCE LISTING (1 sequence)

SEQ ID NO: 1 - protein, 210 aa, Artificial Sequence
  Synthesized Bacillus stearothermophilus Sequence
MTNVLYITAHPHDDTQSYSMAVGKAFIDTYKQVHPDHEVIHLDLYKEYIPEIDVDVFSGWGKLRSGKSFEELSDEEKAKVGRMNELCEQFISADKYVFVTPMWNSFPPVLKAYIDAVAVAGKTFKYTEQGPVGLLTDKKALHIQARGGFYSEGPAAEMEMGHRYLSVIMQFFGVPSFEGLFVEGHAAVPEKAEEIKANAIARAKDLAHTF